(19) United States
(12) Patent Application Publication
(10) Pub. No.: US 2024/0241347 A1
(43) Pub. Date: Jul. 18, 2024

(54) COMPOUND LENS

(71) Applicant: OmniVision Technologies, Inc., Santa Clara, CA (US)

(72) Inventors: I-Lung LU, New Taipei City (TW); Jau-Jan DENG, Taipei (TW); Kuang-Ju WANG, Taipei City (TW)

(21) Appl. No.: 18/098,649

(22) Filed: Jan. 18, 2023

(51) Int. Cl. G02B 13/00; G02B 9/34

(52) U.S. Cl. CPC G02B 13/004; G02B 9/34

(57) ABSTRACT

A compound lens includes four coaxially aligned lenses: first lens and, in order of increasing distance therefrom and on a same side thereof, a second lens, a third lens, and a fourth lens. The first lens and the third lens are negative lenses. The second lens and the fourth lens are positive lenses. An image-side surface of the second lens and an object-side surface of the third lens have respective radii of curvature of equal magnitude.

### BACKGROUND

Medical endoscopy, machine vision, eye/face tracking, and other applications require a compact camera that is able to capture a quality image with a wide field-of-view, and is manufacturable via a low-cost process compatible with high-volume manufacturing.

### SUMMARY OF THE EMBODIMENTS

Embodiments disclosed herein include lenses that enable such a camera. A compound lens includes four coaxially aligned lenses: a first lens and, in order of increasing distance therefrom and on a same side thereof, a second lens, a third lens, and a fourth lens. The first lens and the third lens are negative lenses. The second lens and the fourth lens are positive lenses. An image-side surface of the second lens and an object-side surface of the third lens have respective radii of curvature of equal magnitude.

### BRIEF DESCRIPTION OF THE FIGURES

FIG. 1 is a cross-sectional view of a ventricle that includes a lesion imaged by an endoscope camera that includes a compound lens, in an embodiment.

FIG. 2 is a schematic cross-sectional view of a compound lens, which is an embodiment of the compound lens of FIG. 1.

FIG. 3 is a cross-sectional view of an embodiment of the compound lens of FIG. 2 for visible-light imaging.

FIG. 4 shows a table of example parameters of the compound lens of FIG. 3.

FIG. 5 is a cross-sectional view of an embodiment of the compound lens of FIG. 2 for infrared imaging.

FIG. 6 shows a table of example parameters of the compound lens of FIG. 5.

FIG. 7 is a cross-sectional view of an embodiment of the compound lens of FIG. 2 for applications requiring an athermal lens.

FIG. 8 shows a table of example parameters of the compound lens of FIG. 7.

### DETAILED DESCRIPTION OF THE EMBODIMENTS

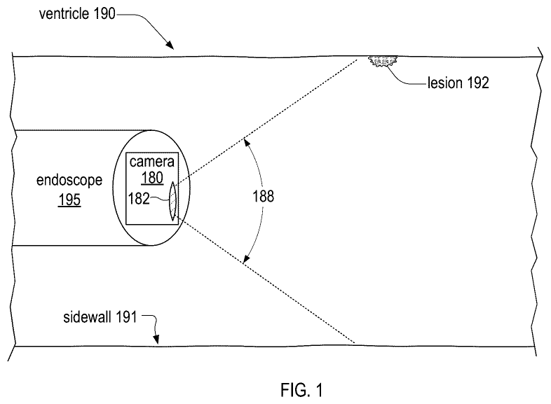

FIG. 1 is a cross-sectional view of an endoscope 195 inside a ventricle 190 that includes a lesion 192. Lesion 192 is on a ventricle sidewall 191. Ventricle 190 may be, for example, a portion of an esophagus or an intestine. Endoscope 195 includes a camera 180, which images lesion 192. Camera 180 includes a lens 182, which in part determines of a field of view 188 of camera 180.

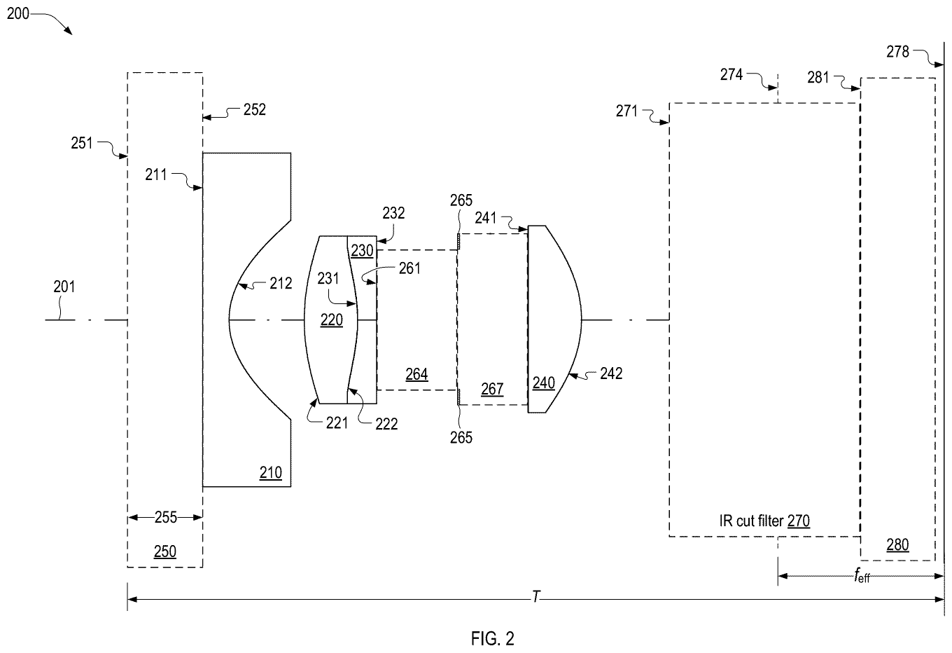

FIG. 2 is a cross-sectional view of a compound lens 200, which is an example of lens 182 of camera 180. Compound lens 200 includes a lens 210 and, in order of increasing distance therefrom and on a same side thereof, a lens 220, a lens 230, and a lens 240. Lens 210 and lens 230 are negative lenses. Lens 220 and lens 240 are positive lenses. Lenses 210-240 are coaxial about a common optical axis 201. Lenses 210-240 have respective object-side surfaces 211, 221, 231, and 241, and respective image-side surfaces 212, 222, 232, and 242. At least one of surfaces 211, 212, 221, 222, 231, 232, 241, and 242 may be aspheric.

Image-side surface 222 and object-side surface 231 may have respective radii of curvature of equal magnitude. In embodiments, at optical axis 201, image-side surface 222 is convex and object-side surface 231 is concave. Lenses 220 and 230 may be respective layers of a layered lens, in which case image-side surface 222 is at least one of conformal to and adjoining object-side surface 231. Compound lens 200 may include an adhesive layer between, and conformal to, each of image-side surface 222 and object-side surface 231.

Compound lens 200 may include at least one of a biplanar substrate 250, a biplanar substrate 264, a biplanar substrate 267, an IR-cut filter 270, and a cover glass 280. Biplanar substrate 264, IR-cut filter 270, and cover glass 280 have respective object-side surfaces 261, 271 and 281. Biplanar substrate 250 has an object-side surface 251 and an image-side surface 252. Each of object-side surfaces 211 and 241 may be planar, and be on surface 252 and an image-side surface of biplanar substrate 267, respectively. Image-side surface 232 may be planar and be on object-side surface of biplanar substrate 264. Biplanar substrate 250 has a thickness 255, which may be between 0.1 millimeters and 0.6 millimeters. Compound lens 200 has an aperture stop 265, which may be between substrates 264 and 267, e.g., at the object-side surface of substrate 267 as shown in FIG. 2.

Compound lens 200 has an effective focal length f_{eff} between a principal plane 274 and an image plane 278, and a total track length T between object-side surface 251 and image plane 278. In embodiments, the ratio T/f_{eff} satisfies 3.4<T/f_{eff}<8.4, which ensures that a transverse dimension of compound lens 200, along a direction perpendicular to optical axis 201, is less than an upper limit. An example of this upper limit is two millimeters.

Lenses 210, 220, 230, and 240 have respective focal lengths f₁, f₂, f₃, and f₄. In embodiments, the ratio f₂/f_{eff} satisfies 0.7<f₂/f_{eff}<2. A benefit of limiting the ratio f₂/f_{eff} to the aforementioned range is to balance aberrations such as coma and distortion of compound lens 200. The ratio f₁/f₄ may satisfy −0.7<f₁/f₄<−0.4, which limits astigmatism and field curvature of compound lens 200.

Lenses 220 and 230 have respective Abbe numbers V₂₂₀ and V₂₃₀, which are computed at the blue, green, and red Fraunhofer F-, d- and C-spectral lines: λ_{F}=486.1 nm, λ_{d}=587.6 nm, and λ_{c}=656.3 nm, respectively. In embodiments, |V₂₂₀−V₂₃₀|≥20, which results in reduced chromatic aberrations such as lateral color and axial color. V₂₂₀ may exceed V₂₃₀.

Lens 220 and lens 230 have respective refractive indices n₂ and n₃, and respective temperature dependences Δn₂/ΔT and Δn₃/ΔT. In embodiments, the ratio (Δn₂/ΔT)/(Δn₃/ΔT) satisfies 0.3<(Δn₂/ΔT)/(Δn₃/ΔT)<1.2, such that the effective focal length f_{eff} of compound lens 200 changes by less than 0.3 micrometers per degree Celsius over a temperature range. An example of this temperature range is −20° C. to 70° C. The refractive indices n₁ and n₂ may be at a visible wavelength (380 nm to 750 nm) or at a near-infrared wavelength (750 nm to 2500 nm).

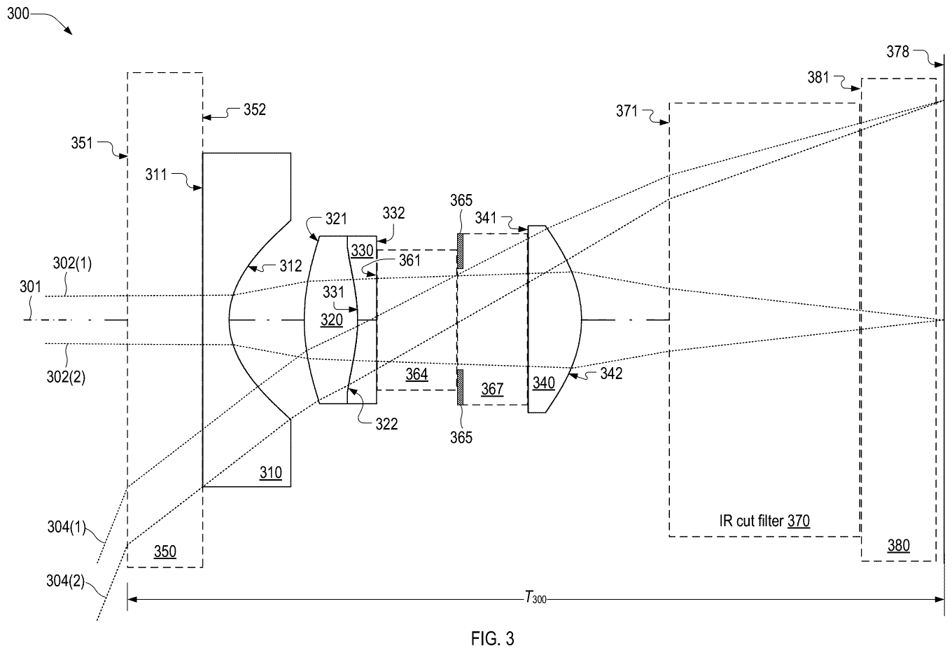

FIG. 3 is a cross-sectional view of a compound lens 300, which is an embodiment of compound lens 200 for imaging at visible wavelengths. Compound lens 300 includes lenses 310, 320, 330, and 340, and may also include at least one of biplanar substrates 350, 364, and 367, an aperture stop 365, an IR-cut filter 370, and a cover glass 380. Lenses 310-340 are coaxial about an optical axis 301. Aperture stop 365 is between substrates 364 and 367. FIG. 3 illustrates compound lens 300 focusing two pairs of parallel optical rays—ray pair 302(1,2) and ray pair 304(1,2)—to respective locations on a focal plane 378.

Lenses 310-340 have respective object-side surfaces 311, 321, 331, and 341, and respective image-side surfaces 312, 322, 332, and 342. Substrates 350, 364, IR-cut filter 370, and cover glass 380 have respective object-side surfaces 351, 361, 371, and 381. Substrate 350 has an image-side surface 352.

Herein, an element of FIG. 3 or subsequent figures denoted by a reference number with a specific tens-place value and ones-place value is an example of the element of FIG. 2 having the same tens-place value and ones-place value. For example, lens 310, surface 311, and surface 312 are respective examples of lens 210, surface 211, and surface 212.

Image-side surface 322 of lens 320 is conformal to, and adjoins, object-side surface 331 of lens 330, such that surfaces 322 and 331 are in direct contact at both zero and non-zero radial distances from optical axis 201. Compound lens 300 includes no layers between surfaces 322 and 331.

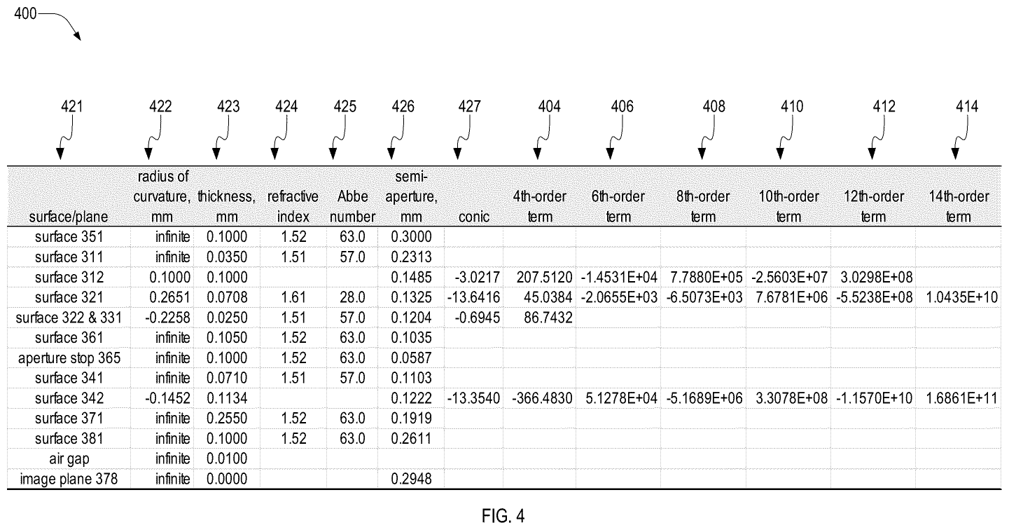

FIG. 4 is a table 400 of example parameters of surfaces and substrates of compound lens 300. Table 400 includes columns 404, 406, 408, 410, 412, 414 and 421-427. Column 421 denotes surfaces of compound lens 300, and also aperture stop 365.

Column 423 includes thickness values between adjacent surfaces of compound lens 300 on optical axis 301. For example, the axial distance between surfaces 311 and 312 is 0.035 millimeters, which is the axial thickness of lens 310. Column 426 indicates the minimum diameter of each surface sufficient for a ray incident on surface 311 that passes through aperture stop 365 to also pass through that surface.

Non-planar surfaces of table 400 are defined by surface sag z_{sag}, shown in Eqn. 1.

$${z}_{s a g} = \frac{{R}^{- 1} {r}^{2}}{1 + \sqrt{1 - ( 1 + k ) {R}^{- 2} {r}^{2}}} + {\sum }_{i = 2}^{N} {α}_{2 i} {r}^{2 i} \qquad ( 1 )$$

In Eqn. 1, z_{sag} is a function of radial coordinate r, where directions z and r are, respectively, parallel to and perpendicular to, optical axis 201. Index i is a positive integer and, in the example of FIG. 4, N=7. In Eqn. 1, the parameter R is the surface radius of curvature, listed in column 422 of table 400. Parameter k denotes the conic constant, shown in column 427. Columns 404, 406, 408, 410, 412, and 414 contain values of aspheric coefficients α₄, α₆, α₈, α₁₀, α₁₂, and α₁₄ respectively. The units of quantities in table 400 are consistent with z_{sag} in Eqn. 1 being expressed in millimeters. Since surfaces 322 and 331 are conformal, they have the same surface sag.

Columns 424 and 425 list values of material refractive index, at free-space wavelength λ_{d}=587.6 nm, and Abbe number, respectively. The refractive index and Abbe number corresponding to a surface characterizes the material between the surface and the surface in the row beneath. For example, the refractive index and Abbe number associated with surface 311 are 1.51 and 57, which are the refractive index and Abbe number of lens 310, respectively.

Compound lens 300 has an effective focal length f₃₀₀=0.20 mm, a field of view of 140 degrees, and an f-number equal to 2.8. The total track length of compound lens 300 is T₃₀₀=1.085 mm between surface 311 and image plane 378. The ratio of total track length to effective focal length is T₃₀₀/f₃₀₀=5.4.

Lenses 310-340 have respective focal lengths f₁, f₂, f₃, and f₄, each which may be approximated by the lensmaker's equation using values of radii of curvature, axial thickness, and refractive index from Table 4. The computed focal lengths are f₁=−0.20 mm, f₂=0.21 mm, and f₄=0.29 mm, such that f₁/f₄=−0.69 and f₂/f₃₀₀=1.06. Focal length f₃=−0.44 mm.

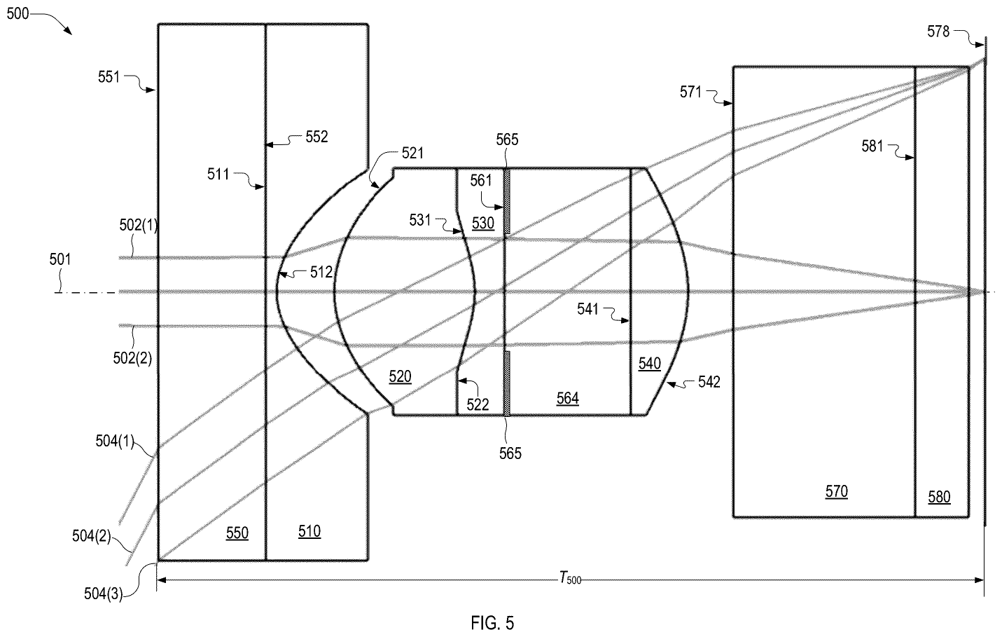

FIG. 5 is a cross-sectional view of a compound lens 500, which is an embodiment of compound lens 200 for infrared imaging applications. Compound lens 500 includes lenses 510, 520, 530, and 540, and may also include at least one of biplanar substrates 550, 567, an aperture stop 565, an IR-pass filter 570, and a cover glass 580. Lenses 510-540 are coaxial about an optical axis 501. Aperture stop 565 is between lens 530 and substrate 764. FIG. 5 illustrates compound lens 500 focusing two sets of parallel optical rays—ray pair 502(1,2) and ray triad 504(1,2,3)—to respective locations on an image plane 578.

Lenses 510-540 have respective object-side surfaces 511, 521, 531, and 541, and respective image-side surfaces 512, 522, 532, and 542. Substrates 550, 564, IR-pass filter 570, and cover glass 580 have respective object-side surfaces 551, 561, 571, and 581. Substrate 550 has an image-side surface 552.

Image-side surface 522 of lens 520 is conformal to, and adjoins, object-side surface 531 of lens 530, such that surfaces 522 and 531 are in direct contact at both zero and non-zero radial distances from optical axis 501. Compound lens 500 includes no layers between surfaces 522 and 531.

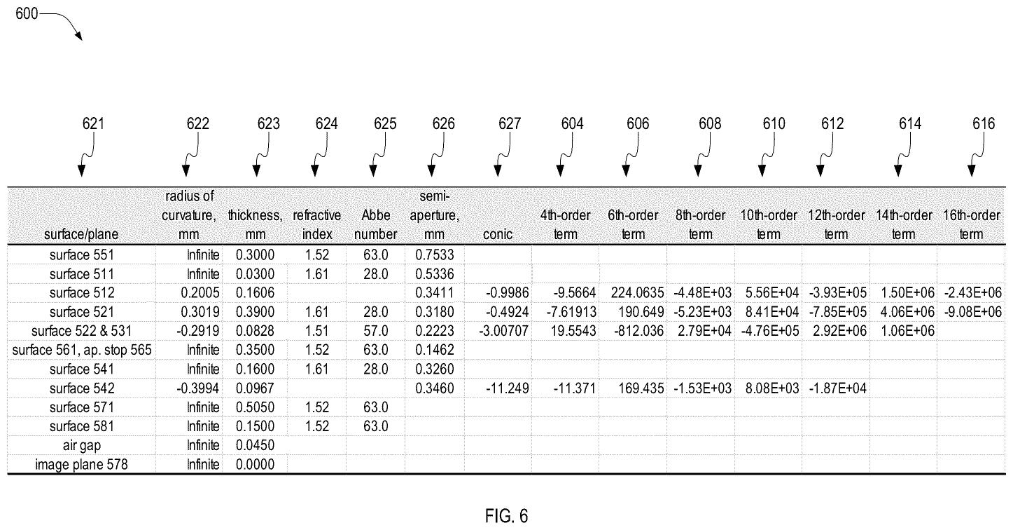

FIG. 6 is a table 600 of example parameters of surfaces and substrates of compound lens 500. Table 600 includes columns 604, 606, 608, 610, 612, 614 and 621-627, which follow the same convention of Table 400 described above. Column 621 denotes surfaces of compound lens 500, and also aperture stop 565. Columns 624 and 625 are analogous to columns 424 and 425 of Table 400, and therefor include values of material refractive index, at free-space wavelength λ_{d}=587.6 nm, and Abbe number, respectively.

Compound lens 500 has an effective focal length f₅₀₀=0.42 mm, a field of view of 130 degrees, and an f-number equal to 2.2. The total track length of compound lens 500 is T₅₀₀=2.3 mm between surface 511 and image plane 578. The ratio of total track length to effective focal length is T₅₀₀/f₅₀₀=5.5.

Lenses 510-540 have respective focal lengths f₁, f₂, f₃, and f₄, each which may be approximated by the lensmaker's equation using values of radii of curvature, axial thickness, and refractive index from Table 6. The computed focal lengths are f₁=−0.33 mm, f₂=0.32 mm, and f₄=0.66 mm, such that f₁/f₄=−0.50 and f₂/f₅₀₀=0.77. Focal length f₃=−0.57 mm.

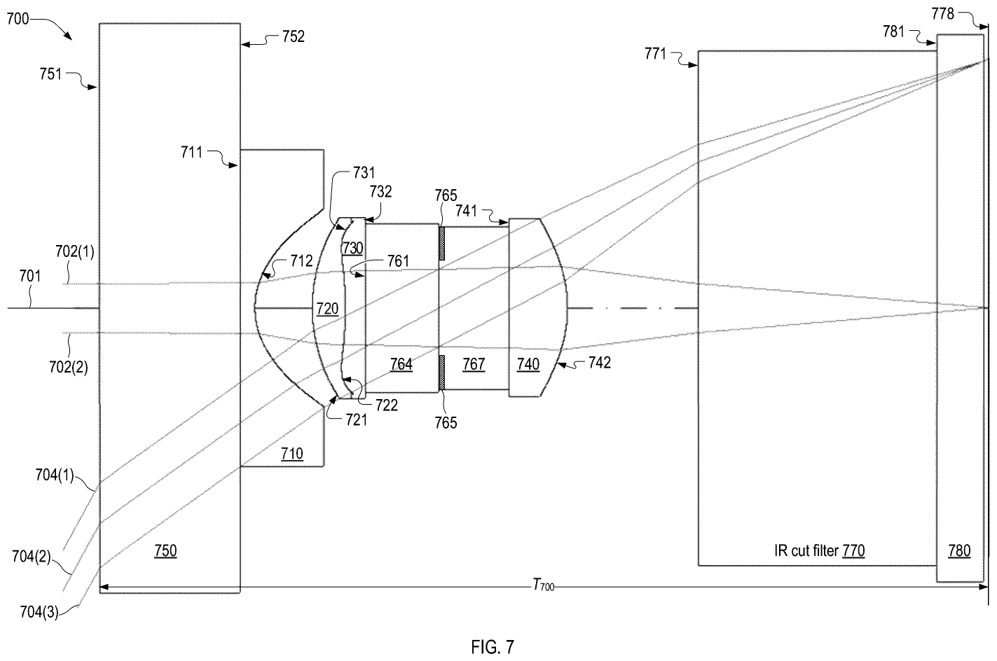

FIG. 7 is a cross-sectional view of a compound lens 700, which is an embodiment of compound lens 200 for applications requiring an athermal lens. Compound lens 700 includes lenses 710, 720, 730, and 740, and may also include at least one of biplanar substrates 750, 764, and 767, an aperture stop 765, an IR-cut filter 770, and a cover glass 780. Aperture stop 765 is between substrates 764 and 767. Lenses 710-740 are coaxial about an optical axis 701. FIG. 7 illustrates compound lens 700 focusing two sets of parallel optical rays—ray pair 702(1,2) and ray triad 704(1,2,3)—to respective locations on an image plane 778.

Lenses 710-740 have respective object-side surfaces 711, 721, 731, and 741, and respective image-side surfaces 712, 722, 732, and 742. Substrates 750, 764, and cover glass 780 have respective object-side surfaces 751, 761, 771, and 781. Substrate 750 has an image-side surface 752.

Image-side surface 722 of lens 720 is conformal to, and adjoins, object-side surface 731 of lens 730, such that surfaces 722 and 731 are in direct contact at both zero and non-zero radial distances from optical axis 201. Compound lens 700 includes no layers between surfaces 722 and 731.

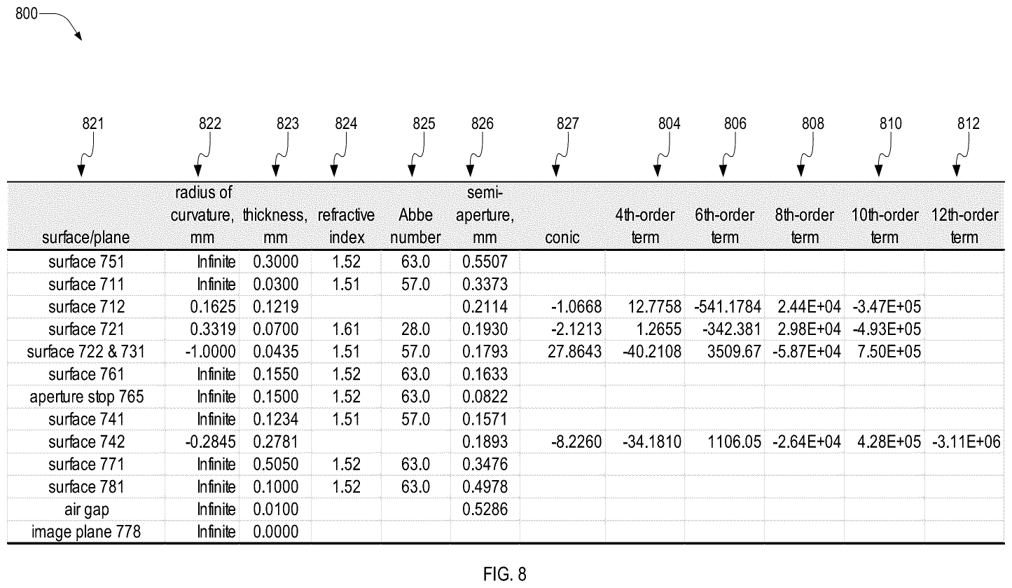

FIG. 8 is a table 800 of example parameters of surfaces and substrates of compound lens 700. Table 800 includes columns 804, 806, 808, 810, 812, and 821-827, which follow the same convention of Table 400 described above. Column 821 denotes surfaces of compound lens 700, and also aperture stop 765. Columns 824 and 825 are analogous to columns 424 and 425 of Table 400, and list values of material refractive index, at free-space wavelength λ_{d}=587.6 nm, and Abbe number, respectively.

Compound lens 700 has an effective focal length f₇₀₀=0.43 mm, a field of view of 125 degrees, and an f-number equal to 4.0. The total track length of compound lens 700 is T₇₀₀=1.89 mm between surface 711 and image plane 778. The ratio of total track length to effective focal length is T₇₀₀/f₇₀₀=4.4.

Lenses 710-740 have respective focal lengths f₁, f₂, f₃, and f₄, each which may be approximated by the lensmaker's equation using values of radii of curvature, axial thickness, and refractive index from Table 8. The computed focal lengths are f₁=−0.32 mm, f₂=0.42 mm, and f₄=0.56 mm, such that f₁/f₄=−0.57 and f₂/f₇₀₀=0.97. Focal length f₃=−1.96 mm.

### Combinations of Features

Features described above as well as those claimed below may be combined in various ways without departing from the scope hereof. The following enumerated examples illustrate some possible, non-limiting combinations:
(A1) A compound lens includes four coaxially aligned lenses: a first lens and, in order of increasing distance therefrom and on a same side thereof, a second lens, a third lens, and a fourth lens. The first lens and the third lens are negative lenses. The second lens and the fourth lens are positive lenses. An image-side surface of the second lens and an object-side surface of the third lens have respective radii of curvature of equal magnitude.
(A2) In embodiments of (A1), the image-side surface is conformal to the object-side surface.
(A3) In either of embodiments (A1) or (A2), the image-side surface adjoins the object-side surface.
(A4) Any of embodiments (A1)-(A3) further includes an adhesive layer between, and conformal to, each of the image-side surface and the object-side surface.
(A5) In any of embodiments (A1)-(A4), the image-side surface and the object-side surface are convex and concave, respectively, at the optical axis of the four coaxially aligned lenses.
(A6) Any of embodiments (A1)-(A5) further includes a first biplanar substrate, the first lens having a planar object-side surface on the image-side surface of the first biplanar substrate.
(A7) In embodiment (A6), a thickness of the first biplanar substrate is between 0.1 millimeters and 0.6 millimeters.
(A8) In any of embodiments (A1)-(A7), the first lens, second lens, third lens, and fourth lens collectively have an effective focal length f_{eff} such that the image is formed at an image plane located a distance T from an object-side surface of the first biplanar substrate, and the ratio T/f_{eff} satisfying 3.4<T/f_{eff}<8.4.
(A9) Any of embodiments (A1)-(A8) further includes a second biplanar substrate having a planar object-side surface. The third lens has a planar image-side surface on the planar object-side surface.
(A10) In any of embodiments (A1)-(A9), the first lens, second lens, third lens, and fourth lens collectively have an effective focal length f_{eff} such that an image is formed at an image plane.
(A11) In embodiment (A10), the second lens has a focal length f₂, and the ratio f₂/f_{eff} satisfies 0.7<f₂/f_{eff}<2.
(A12) In any of embodiments (A1)-(A11), the first lens and the fourth lens having respective focal lengths f₁ and f₄, the ratio f₁/f₄ satisfies −0.7<f₁/f₄<−0.4.
(A13) In any of embodiments (A1)-(A12), a difference between respective Abbe numbers of the second lens and the third lens being greater than or equal to twenty.
(A14) In any of embodiments (A1)-(A13), the second lens and the third lens have respective refractive indices n₂ and n₃ that have respective temperature dependences Δn₂/ΔT and Δn₃/ΔT. The ratio (Δn₂/ΔT)/(Δn₃/ΔT) satisfies 0.3<(Δn₂/ΔT)/(Δn₃/ΔT)<1.2.

Changes may be made in the above methods and systems without departing from the scope of the present embodiments. It should thus be noted that the matter contained in the above description or shown in the accompanying drawings should be interpreted as illustrative and not in a limiting sense. Herein, and unless otherwise indicated, the phrase “in embodiments” is equivalent to the phrase “in certain embodiments,” and does not refer to all embodiments. The following claims are intended to cover all generic and specific features described herein, as well as all statements of the scope of the present method and system, which, as a matter of language, might be said to fall therebetween.

What is claimed is:

1. A compound lens comprising: four coaxially aligned lenses including a first lens and, in order of increasing distance therefrom and on a same side thereof, a second lens, a third lens, and a fourth lens;
the first lens and the third lens being negative lenses;
the second lens and the fourth lens being positive lenses;
an image-side surface of the second lens and an object-side surface of the third lens having respective radii of curvature of equal magnitude.

2. The compound lens of claim 1, the image-side surface being conformal to the object-side surface.

3. The compound lens of claim 1, the image-side surface adjoining the object-side surface.

4. The compound lens of claim 1, further comprising an adhesive layer between, and conformal to, each of the image-side surface and the object-side surface.

5. The compound lens of claim 1, the image-side surface and the object-side surface being convex and concave, respectively, at the optical axis of the four coaxially aligned lenses.

6. The compound lens of claim 1, further comprising a first biplanar substrate, the first lens having a planar object-side surface on the image-side surface of the first biplanar substrate.

7. The compound lens of claim 6, a thickness of the first biplanar substrate being between 0.1 millimeters and 0.6 millimeters.

8. The compound lens of claim 6, the first lens, second lens, third lens, and fourth lens collectively having an effective focal length f_{eff} such that the image is formed at an image plane located a distance T from an object-side surface of the first biplanar substrate, and the ratio T/f_{eff} satisfying 3.4<T/f_{eff}<8.4.

9. The compound lens of claim 1, further comprising a second biplanar substrate having a planar object-side surface, the third lens having a planar image-side surface on the planar object-side surface.

10. The compound lens of claim 1, the first lens, second lens, third lens, and fourth lens collectively having an effective focal length f_{eff} such that an image is formed at an image plane.

11. The compound lens of claim 10, the second lens having a focal length f₂, the ratio f₂/f_{eff} satisfying 0.7<f₂/f_{eff}<2.

12. The compound lens of claim 1, the first lens and the fourth lens having respective focal lengths f₁ and f₄, the ratio f₁/f₄ satisfying −0.7<f₁/f₄<−0.4.

13. The compound lens of claim 1, a difference between respective Abbe numbers of the second lens and the third lens being greater than or equal to twenty.

14. The compound lens of claim 1, the second lens and the third lens have respective refractive indices n₂ and n₃ that have respective temperature dependences Δn₂/ΔT and Δn₃/ΔT, the ratio (Δn₂/ΔT)/(Δn₃/ΔT) satisfying 0.3<(Δn₂/ΔT)/(Δn₃/ΔT)<1.2.
